(19) United States
(12) Patent Application Publication
(10) Pub. No.: US 2021/0071228 A1
(43) Pub. Date: Mar. 11, 2021

(54) METHOD FOR DETECTING GUT MICROORGANISM IN A SAMPLE USING NORMAL GUT FLORA AS INTERNAL CONTROL

(71) Applicant: SEEGENE, INC., Seoul (KR)

(72) Inventors: Hyeon Ji LEE, Seoul (KR); Myung Suk OH, Seoul (KR); Tommaso GIANI, Siena (IT); Fabio ARENA, Siena (IT); Alberto ANTONELLI, Siena (IT); Gian Maria ROSSOLINI, Siena (IT)

(21) Appl. No.: 17/054,233

(22) PCT Filed: May 10, 2018
(86) PCT No.: PCT/KR2018/005338

§ 371 Date: Nov. 10, 2020

(51) Int. Cl. C12Q 1/06; C12Q 1/6806; C12Q 1/6851; C12Q 3/00

(52) U.S. Cl. CPC C12Q 1/06; C12Q 1/6806; C12Q 2531/113; C12Q 3/00; C12Q 2500/00; C12Q 1/6851

(57) ABSTRACT

The present invention relates to a method for detecting a nucleic acid of a gut microorganism in a sample using a nucleic acid of a bacterium as an internal control nucleic acid selected from a normal gut flora, and to a composition for nucleic acid amplification used in the method. The internal control according to the present invention is present in the sample from the beginning, and thus there is no inconvenience of separately adding an internal control after the sample collection process, and may be used as an internal control for the sample collection process, an internal control for the nucleic acid extraction process, and an internal control for the nucleic acid amplification process. In addition, the presence or absence of the nucleic acid of the gut microorganism in the sample may be detected with a high accuracy through the minimization of false negative and false-positive determinations by using the nucleic acid of the bacterium as the internal control selected from the normal gut flora.

### TECHNICAL FIELD

The present invention relates to a method for detecting a nucleic acid of a gut microorganism in a sample using a nucleic acid of a bacterium as an internal control nucleic acid selected from a normal gut flora, and to a composition for nucleic acid amplification used in the method.

### BACKGROUND ART

Human diseases may be detected through in vivo diagnosis and in vitro diagnosis. The causes of diseases are analyzed through X-ray and CT scans and the like for in vivo diagnosis and through urine, blood, histiocytes, and the like for in vitro diagnosis.

In vitro diagnosis includes immunochemical diagnosis, self-monitoring of blood glucose, field diagnosis, molecular diagnosis, and the like. Of these, molecular diagnosis is a technique for directly testing genes using PCR or the like corresponding to gene amplification, wherein it is investigated whether persons are infected with a disease by extracting nucleic acids containing gene information of pathogens from samples, such as saliva, blood, and feces of persons infected with viruses or bacteria and then amplifying the extracted nucleic acids. Such molecular diagnosis has the advantages of being more accurate than blood or urine tests and not having to take a biopsy, and enables prevention and efficient treatment of diseases through early diagnosis.

In the molecular diagnosis, the collection of suitable and accurate samples for tests is essential for accurate test results. In the collection of samples, insufficient sample volumes or contaminated samples may cause inaccurate test results, increasing a recall rate and delaying the reporting of test results. As described above, the molecular diagnosis encompasses the processes of extracting nucleic acids from samples and amplifying the extracted nucleic acids, and when a loss of nucleic acids occurs in the nucleic acid extraction process or a material to inhibit an amplification reaction (e.g., heparin, a surfactant, a protein denaturant, or an organic solvent) is contained in sample solutions, amplification efficiency is lowered, so that nucleic acids of pathogens cannot be sufficiently amplified. In this case, despite the presence of pathogens in the samples, the nucleic acids of the pathogens cannot be amplified, causing false-negative results, and even the same samples may yield different results depending on the degree of loss in the nucleic acid extraction process.

In order to solve the problems, a method of confirming false negatives using an internal control for the processes of extracting nucleic acids from samples and amplifying the extracted nucleic acids was developed (U.S. Pat. No. 5,770,360). A nucleic acid that is usually present in a sample or is added to the sample prior to the extraction of nucleic acids is used as an internal control, so that the nucleic acid of the control is usually extracted, amplified, and detected regardless of the presence of pathogens in the sample. The non-detection of the nucleic acid of the internal control can confirm that there was a false negative due to a loss in the extraction process or the deterioration in the amplification by a PCR inhibitory material.

There are tens of thousands of species of microorganisms in the guts of humans, and each individual has hundreds of species of microorganisms. These gut microorganisms are shown to have a great influence on digestion, secretion, and the like while having a symbiotic relationship with people over the course of their lives. In recent years, the gut′ microorganisms have been understood as having a profound effect on human physical and mental health and lead to the development of the theory of Gut-Brain Axis, and studies on the gut microorganisms are actively being conducted (Augusto J. et al., Front. Integr. Neurosci., 2013). It has also been proved in various aspects, such as physiology, immunology, and epidemiology, that gut microorganisms are closely associated with autoimmune diseases, such as atopy and asthma, and mental diseases, such as autism and depression. For the above reasons, there is a growing demand for detection and analysis of gut microorganisms, and therefore, the development of an internal control for the detection of gut microorganisms is also needed.

Throughout the specification, many cited documents and patent documents are referenced and their citations are represented. The disclosures of cited documents and patent documents are entirely incorporated by reference into the present specification, so that the level of the technical field within which the present invention falls, and the details of the present invention are explained more clearly.

### DETAILED DESCRIPTION OF THE INVENTION

### Technical Problem

The present inventors researched and endeavored to develop a novel internal control capable of improving the accuracy of detection by minimizing false-negative and false-positive determinations in the gut microorganism detection method using nucleic acid amplification.

As a result, the present inventors experimentally verified that a nucleic acid of a bacterium selected from a normal gut flora can be successfully used as an internal control for the processes of collecting a sample, extracting nucleic acids from the sample, and/or amplifying the extracted nucleic acids in the gut microorganism detection method using nucleic acid amplification, and thus completed the present invention.

Therefore, an aspect of the present invention is to provide a method for detecting a nucleic acid of a gut microorganism in a sample using a nucleic acid of a bacterium as an internal control nucleic acid selected from a normal gut flora.

Another aspect of the present invention is to provide a composition for amplifying a nucleic acid of a gut microorganism in a sample using a nucleic acid of a bacterium as an internal control nucleic acid selected from a normal gut flora.

Other purposes and advantages of the present invention will be clarified by the following detailed description of the invention, claims, and drawings.

### Technical Solution

In accordance with an aspect of the present invention, there is provided a method for detecting a nucleic acid of a gut microorganism in a sample using a nucleic acid of a bacterium as an internal control nucleic acid selected from a normal gut flora, the method including:

collecting and preparing the sample;

performing an amplification reaction of a nucleic acid in the sample using (i) a pair of primers for amplifying the nucleic acid of the gut microorganism; and (ii) a pair of primers for amplifying the nucleic acid of the bacterium as the internal control nucleic acid selected from the normal gut flora;

detecting a resultant of the amplification reaction;

determining a validity of the amplification reaction of the nucleic acid of the gut microorganism by using a resultant of the amplification reaction of the internal control nucleic acid; and

determining whether the nucleic acid of the gut microorganism is present or not in the sample by (i) the determined validity and (ii) the resultant of the amplification reaction of the nucleic acid of the gut microorganism.

The present inventors researched and endeavored to develop a novel internal control usable in the gut microorganism detection method using nucleic acid amplification. As a result, the present inventors established a novel protocol capable of detecting a nucleic acid of a gut microorganism in a sample using a nucleic acid of a bacterium as an internal control nucleic acid selected from a normal gut flora present in a gut, and according to the novel protocol, the nucleic acid of the bacterium selected from the normal gut flora is used as an internal control for the processes of collecting a sample, extracting nucleic acids from the sample, and/or amplifying the extracted nucleic acids.

Hereinafter, the present invention will be described in more detail as follows:

Collecting and Preparing Sample

According to the present invention, a sample is first collected and prepared.

As used herein, the term “sample” refers to a sample obtained from a human or animal subject, of which a nucleic acid of a gut microorganism is to be detected by nucleic acid amplification.

According to an embodiment of the present invention, the sample includes, but is not limited to, a rectal swab sample, a stool sample, or a urine sample from a human or an animal. The sample may also include environmental samples from which a nucleic acid of a gut microorganism may be detected, for example, samples collected from toilet bowls, towels, toilet paper, and the like, with which a human or an animal has come into contact.

According to an embodiment of the present invention, the sample is one obtained from a human and animal subject suspected of having a pathogenic microorganism infection.

According to an embodiment of the present invention, the animal includes, but is not limited to, primates, livestock (e.g., pigs, sheep, cows, horses, and donkeys), experimental animals (e.g., rats, mice, guinea pigs, hamsters, and rabbits), pets (e.g., dogs and cats), domesticated wild animals (e.g., squirrels, foxes, kangaroos, and deer), and birds.

According to an embodiment of the present invention, nucleic acids in the sample may be directly analyzed without a step of extracting nucleic acids from the sample. For example, the methods disclosed in Pannacio et al. (Nucleic Acids Res. 1993 Sep. 25; 21(19): 4656) and Pandori et al. (BMC Infect Dis. 2006 Jun. 24; 6: 104) may be used.

According to an embodiment of the present invention, the preparation of the sample further includes a step of extracting nucleic acids from the sample.

As used herein, the term “nucleic acid” or “nucleic acid molecule” refers to a single-stranded form or double-stranded form of deoxyribonucleotide or ribonucleotide polymer, and the nucleotides include derivatives of naturally occurring nucleotides, non-naturally occurring nucleotides, or modified nucleotides, all of the nucleotides being capable of functioning in the same manner as naturally occurring nucleotides.

The extraction of the nucleic acids from the sample may employ various methods known in the art, and a specific method therefor is disclosed in Sambrook et al., Molecular Cloning, A Laboratory Manual, Cold Spring Harbor Laboratory Press (2001). In addition, various kits for nucleic acid extraction according to the kind of sample are commercially available, and those skilled in the art can extract nucleic acids from various samples using commercially available kits.

Amplification Reaction of Nucleic Acid in Sample

According to the present invention, the nucleic acids in the sample are amplified using (i) a pair of primers for amplifying the nucleic acid of the gut microorganism; and (ii) a pair of primers for amplifying the nucleic acid of the bacterium as the internal control nucleic acid selected from the normal gut flora.

The amplification reaction of the nucleic acids may further include (i) a probe for detecting the nucleic acid of the gut microorganism; and (ii) a probe for detecting the nucleic acid of the bacterium selected from the normal gut flora.

As used herein, the term “primer” refers to an oligonucleotide, which acts as a point of initiation of synthesis under conditions in which the synthesis of primer extension products complementary to a nucleic acid chain (template) is induced, i.e., the presence of nucleotides and an agent for polymerization, such as nucleic acid polymerase, as well as suitable temperature and pH.

As used herein, the term “probe” refers to a single-stranded nucleic acid molecule including a portion or portions that are substantially complementary to a target nucleic acid sequence.

The primers or probes used in the present invention may include naturally occurring NMPs (i.e., AMP, GMP, CMP, and UMP), naturally occurring dNMPs (i.e., dAMP, dGMP, dCMP, and dTMP), modified nucleotides, or non-naturally occurring nucleotides.

The primers need to be sufficiently long to prime the synthesis of extension products in the presence of the agent for polymerization. The appropriate length of the primers depends on several factors, such as temperature, field of application, and sources of primers.

As used herein, the term “annealing” or “priming” refers to the apposition of an oligonucleotide or nucleic acid to a template nucleic acid, wherein the apposition enables the polymerase to polymerize nucleotides to form a nucleic acid molecule which is complementary to the template nucleic acid or a portion thereof.

As used herein, the term “complementary” is used to mean that primers or probes are sufficiently complementary to hybridize selectively with a target nucleic acid molecule under predetermined annealing or hybridization conditions, and the term encompasses “substantially complementary” and “perfectly complementary”, and specifically means “perfectly complementary”.

As used herein, the term “target nucleic acid”, “target nucleic acid sequence”, or “target sequence molecule” refers to a nucleic acid molecule to be ultimately amplified or detected, and the target nucleic acid is annealed to or hybridizes with primers under particular hybridization conditions.

As used herein, the term “gut microorganism” refers to a microorganism to be detected in the sample.

As used herein, the term “nucleic acid of a gut microorganism” refers to a nucleic acid of a microorganism to be detected in the sample.

According to an embodiment of the present invention, the gut microorganism to be detected through the method of the present invention means a microorganism present in human or animal guts, and the microorganism may include, but is not limited to, bacteria, yeast, fungi, viruses, protozoans, and the like.

According to an embodiment of the present invention, the gut microorganism may be a drug-resistant gut bacterium.

According to an embodiment of the present invention, the drug-resistant gut bacterium may be, but is not limited to, carbapenem-resistant Enterobacteriaceae (CRE), vancomycin-resistant Enterococci (CRE), or extended-spectrum beta-lactamases (ESBL)-producing bacterium.

The nucleic acid of the gut microorganism may include, but is not limited to, a DNA molecule or an RNA molecule.

According to an embodiment of the present invention, the nucleic acid of the drug-resistant gut bacterium may include a resistance gene allowing the exhibition of drug resistance, and may include a gene variation, a resistance gene mediated by, for example, a plasmid, or a transposon.

According to an embodiment of the present invention, the nucleic acid of the bacterium used as the internal control nucleic acid is different from the nucleic acid of the gut microorganism to be detected by the method of the present invention.

According to an embodiment of the present invention, the method for detecting a nucleic acid of a gut microorganism according to the present invention may perform simultaneous detection of 1-30, specifically, 1-25, 1-20, 1-15, 1-10, or 1-5 gut microorganism nucleic acids, and more specifically, 1, 2, 3, 4, 5, 6, 7, 8, 9, 10, 15, 20, 25, or 30 gut microorganism nucleic acids, but is not limited thereto.

According to an embodiment of the present invention, the amplification reaction of the nucleic acids may employ 1-30 pairs, specifically 1-25 pairs, 1-20 pairs, 1-15 pairs, 1-10 pairs, or 1-5 pairs of primers for amplifying nucleic acids of gut microorganisms, and more specifically, 1 pair, 2 pairs, 3 pairs, 4 pairs, 5 pairs, 6 pairs, 7 pairs, 8 pairs, 9 pairs, 10 pairs, 15 pairs, 20 pairs, 25 pairs, or 30 pairs of primers for amplifying nucleic acids of gut microorganisms, but is not limited thereto.

As used herein, the term, “hybridization” refers to the formation of a double-stranded nucleic acid from two single-stranded polynucleotides through non-covalent binding between complementary nucleotide sequences under predetermined hybridization conditions or strict conditions.

The hybridization may occur when two nucleic acid sequences are perfectly complementary (perfect matched) or substantially complementary with some mismatches (e.g., 1-4 mismatches) at a hybridization occurrence site (a double-strand formation site). The degree of complementarity required for hybridization may vary depending on the hybridization reaction conditions, and may be controlled by, particularly, temperature.

As used herein, the terms “hybridization” and “annealing” are not different from each other, and are used interchangeably with each other.

The amplification of a target nucleic acid molecule may be performed by various primer-involved nucleic acid amplification methods known in the art. Specifically, the amplification of a target nucleic acid is performed according to polymerase chain reaction (PCR), which is disclosed in U.S. Pat. Nos. 4,683,195, 4,683,202, and 4,800,159. Other examples are ligase chain reaction (LCR, U.S. Pat. Nos. 4,683,195 and 4,683,202; and PCR Protocols: A Guide to Methods and Applications (Innis et al., eds, 1990)), strand displacement amplification (SDA, Walker, et al. Nucleic Acids Res. 20(7):1691-6 (1992); and Walker PCR Methods Appl. 3(1):1-6 (1993)), transcription-mediated amplification (Phyffer, et al., J. Clin. Microbiol. 34:834-841 (1996); and Vuorinen, et al., J. Clin. Microbiol. 33:1856-1859 (1995)), nucleic acid sequence-based amplification (NASBA, Compton, Nature 350(6313):91-2 (1991)), rolling circle amplification (RCA, Lisby, Mol. Biotechnol. 12(1):75-99 (1999); and Hatch et al., Genet. Anal. 15(2):35-40 (1999)), and Q-Beta Replicase (Lizardi et al., BiolTechnology 6:1197 (1988)).

Various DNA polymerases may be used in the amplification of nucleic acids of the present invention, and include E. coli DNA polymerase I, thermostable DNA polymerase, and bacteriophage T7 DNA polymerase. Specifically, the DNA polymerase is a thermostable DNA polymerase that may be obtained from various species of bacteria, which include Thermus aquaticus (Taq), Thermus thermophilus, Thermus filiformis, Thermus flavus, Thermus antranikianii, Thermus caldophilus, Thermus chliarophilus, Thermus igniterrae, Thermus lacteus, Thermus oshimai, Thermus ruber, Thermus rubens, Thermus scotoductus, Thermus silvanus, Thermus species Z05, and Thermus species sps 17.

According to an embodiment of the present invention, the amplification of nucleic acids may be performed by carrying out fast PCR.

As used herein, the term “fast PCR” refers to a PCR method in which the rate of PCR is increased compared with a general PCR method. Fast PCR may be achieved by controlling various factors, such as the extension rate of DNA polymerase, the ramp speed of the thermal cycler, and the complexity of the template. For example, fast PCR can be attained using Taq DNA polymerase with a standard extension rate of 2-4 kb per minute, instead of Taq DNA polymerase with a standard extension rate of 1 kb per minute, as is used in general PCR.

As used herein, the term “normal gut flora” refers to the population of all bacteria formed in human or animal guts.

According to an embodiment of the present invention, the normal gut flora is a human normal gut flora.

According to an embodiment of the present invention, the normal gut flora may include, but is not limited to, Bacteroides, Lactobacillus, Escherichia, Klebsiella, Proteus, Streptococcus, Staphylococcus, Pseudomonas, and Clostridium.

According to a specific embodiment of the present invention, the bacterium selected from the normal gut flora may be Bacteroides spp., or Lactobacillus spp.

According to an embodiment of the present invention, the nucleic acid of the bacterium selected from the normal gut flora may include a nucleotide sequence encoding 16s rRNA.

According to an embodiment of the present invention, the nucleic acid of the bacterium selected from the normal gut flora may be used as an internal control for the steps of collecting the sample, extracting the nucleic acids from the sample, and/or amplifying the extracted nucleic acids.

The present inventors, on the basis of the fact that the normal gut flora is present in the gut, designed a method capable of detecting a nucleic acid of a gut microorganism in a sample using a nucleic acid of a bacterium as an endogenous internal control nucleic acid selected from a normal gut flora.

In the present invention, the nucleic acid of the bacterium selected from the normal gut flora is amplified simultaneously with the amplification of the nucleic acid of the gut microorganism in the same reaction container. Here, when the amplification of the nucleic acid of the bacterium selected from the normal gut flora is not detected, such non-detection may indicate that a desired process may not be normally executed in at least one of the steps of collecting the sample, extracting the nucleic acids, and amplifying the nucleic acids in the sample. Therefore, the nucleic acid of the bacterium selected from the normal gut flora may be used as an internal control for the respective steps. The roles as the internal control in the respective steps are described below.

(i) Internal Control in Step of Collecting Sample:

In the case where the sample is not properly collected in the step of collecting the sample, for example, when the amount of the collected sample is too small to detect nucleic acids, the nucleic acid of the gut microorganism may not be detected even though the nucleic acid of the gut microorganism is present in the sample. This may cause false-negative results, and such false-negatives may be determined by checking the presence or absence of the nucleic acid of the bacterium as the internal control selected from the normal gut flora.

(ii) Internal Control in Step of Extracting Nucleic Acids:

In the case where the loss of nucleic acids occurs in the step of extracting the nucleic acids, that is, where the amount of the extracted nucleic acid as a template for an amplification reaction is not sufficient, the efficiency of the amplification reaction deteriorates, resulting in no detection of nucleic acids, causing false-negative results. In this case, the false-negatives may be determined by checking the presence or absence of the nucleic acid of the bacterium as the internal control selected from the normal gut flora.

(iii) Internal Control in Step of Amplifying Nucleic Acids:

In the case where a material that inhibits an amplification reaction (e.g., heparin, a surfactant, a protein denaturant, an organic solvent, or the like) is contained in an amplification reaction solution in the step of amplifying nucleic acids, amplification efficiency is lowered regardless of the presence of a target nucleic acid, resulting in no detection of nucleic acid amplification, causing false-negative results. In this case, the false-negatives may be determined by checking the presence or absence of the nucleic acid of the bacterium as the internal control selected from the normal gut flora.

According to an embodiment of the present invention, the method according to the present invention may further include amplifying the nucleic acid in the collected sample using the pair of primers for amplifying the nucleic acid of the bacterium as the internal control nucleic acid selected from the normal gut flora, whereby the collection of the sample may be determined to be valid or invalid.

According to an embodiment of the present invention, the method according to the present invention may further include amplifying the nucleic acid extracted from the sample using the pair of primers for amplifying the nucleic acid of the bacterium as the internal control nucleic acid selected from the normal gut flora, whereby the extraction may be determined to be valid or invalid.

According to an embodiment of the present invention, the amplification reaction of the nucleic acid is performed in the presence of a label or a labeled oligonucleotide (a labeled primer or a labeled probe), capable of providing a signal depending on the presence of a nucleic acid to be detected.

According to an embodiment of the present invention, the signal may be provided from the label during the amplification of the nucleic acid to be detected or the signal may be provided after the completion of the amplification.

Detecting Resultant of Amplification Reaction

According to an embodiment of the present invention, a resultant of the amplification reaction of nucleic acids may be detected during the amplification of nucleic acids or after the completion of the amplification reaction of nucleic acids.

According to an embodiment of the present invention, the detection of the resultant of the amplification reaction may be performed in a post-amplification detection manner or in a real-time detection manner.

The post-amplification detection manner is a method whereby amplicons are detected after the amplification of nucleic acids. The post-amplification detection manner includes, for example, the separation of amplicons according to size difference (e.g., electrophoresis) or the separation of amplicons through immobilization, but is not limited thereto.

Alternatively, for the post-amplification detection manner, post-PCR melting analysis may be used in which, after the amplification of a target nucleic acid sequence, the fluorescence intensity is monitored while the temperature is raised or lowered in a certain period, and then amplicons are detected by melting profiles (U.S. Pat. Nos. 5,871,908 and 6,174,670, and WO 2012/096523).

The real-time detection manner is a method whereby a target nucleic acid sequence may be detected while the amplification of the target nucleic acid is monitored in real time.

The post-amplification manner or the real-time detection manner may use a label or a labeled oligonucleotide for providing a signal depending on the presence of a nucleic acid to be detected.

According to an embodiment of the present invention, the detection of the amplified nucleic acid may be performed by detecting a signal provided from a label during the amplification of the nucleic acid to be detected or detecting a signal provided after the completion of the amplification of the nucleic acid to be detected.

For example, the detection may be performed using a non-specific fluorescence dye that non-specifically intercalates into a duplex, which is an amplicon of the target nucleic acid sequence.

In addition, a labeled primer or probe that specifically hybridizes with the target nucleic acid sequence may be used.

Examples of methods of using a labeled primer include Sunrise primer method (Nazarenko et al, 2516-2521 Nucleic Acids Research, 1997, v. 25 no. 12, and U.S. Pat. No. 6,117,635), Scorpion primer method (Whitcombe et al, 804-807, Nature Biotechnology v. 17 Aug. 1999, and U.S. Pat. No. 6,326,145), and TSG Primer method (WO 2011/078441).

Examples of methods of using a labeled probe include a molecular beacon method using a dual-labeled probe forming a hair-pin structure (Tyagi et al, Nature Biotechnology v. 14 Mar. 1996), a hybridization probe method using two probes single-labeled with a donor or an acceptor (Bernad et al, 147-148 Clin Chem 2000; 46), a Lux method using a single-labeled oligonucleotide (U.S. Pat. No. 7,537,886), and a TaqMan method using a cleavage reaction of the double-labeled probe by 5′-nuclease activity of DNA polymerase as well as the hybridization of a dual-labeled probe (U.S. Pat. Nos. 5,210,015 and 5,538,848), but are not limited thereto.

In addition, the detection may be performed using a duplex formed depending on the presence of the target nucleic acid sequence. The duplex formed depending on the presence of the target nucleic acid sequence is not an amplicon itself of the target sequence formed by the amplification reaction, and the amount of the duplex increases in proportion to the amplification of the target nucleic acid sequence. The duplex formed depending on the presence of the target nucleic acid sequence may be obtained according to various method, for example, Invader assay (U.S. Pat. Nos. 5,691,142, 6,358,691, and 6,194,149), PTOCE (PTO Cleavage and Extension) method (WO 2012/096523), PCEC (PTO Cleavage and Extension-dependent Cleavage) method (WO 2012/134195), PCE-SH (PTO Cleavage and Extension-dependent Signaling Oligonucleotide Hybridization) method (WO 2013/115442), PCE-SC (PTO Cleavage and Extension-dependent Signaling Oligonucleotide Cleavage) method (WO 2013/157821), PCE-NH (PTO Cleavage and Extension-dependent Non-Hybridization) method (WO 2014/104818), and PCE-IH (PTO Cleavage and Extension-dependent Immobilized Oligonucleotide Hybridization) method (WO 2015/008985), the contents of which are incorporated herein by reference.

In addition, for the real-time detection of a target, a method of detecting at least one target nucleic acid sequence through only a single type of label using signal detection at different temperatures may be employed. The techniques therefor are disclosed in WO 2015/147412, WO 2016/093619, and WO 2016/093620, the contents of which are incorporated herein by reference.

According to an embodiment of the present invention, the amplification reaction may further include (i) a probe for detecting the nucleic acid of the gut microorganism; and (ii) a probe for detecting the nucleic acid of the bacterium selected from the normal gut flora.

According to an embodiment of the present invention, the amplification reaction may include 1-30, specifically, 1-25, 1-20, 1-15, 1-10, or 1-5 probes for detecting a gut microorganism nucleic acid, and more specifically, 1, 2, 3, 4, 5, 6, 7, 8, 9, 10, 15, 20, 25, or 30 probes for detecting a gut microorganism nucleic acid, but is not limited thereto.

In a specific embodiment of the present invention, the pair of primers or the probe for the nucleic acid of the bacterium selected from the normal gut flora may include a nucleotide sequence that specifically hybridizes with a nucleotide sequence selected from the group consisting of the sequence of SEQ ID No: 1, the sequence of SEQ ID No: 2, and complementary sequences thereto (FIG. 1).

As used herein, the term “specifically hybridizing” is used to mean that two or more molecules interact with each other via covalent or non-covalent binding, which may be, for example, the binding of a single-stranded target sequence and a single-stranded nucleotide molecule having a nucleotide sequence complementary to the single-stranded target sequence.

Determining Validity of Amplification Reaction of Nucleic Acid of Gut Microorganism and Determining Presence or Absence of Nucleic Acid of Gut Microorganism

The validity of the amplification reaction of the nucleic acid of the gut microorganism is determined from a resultant of the amplification reaction of the internal control nucleic acid, and the presence or absence of the nucleic acid of the gut microorganism is determined by (i) the determined validity and (ii) the resultant of the amplification reaction of the nucleic acid of the gut microorganism.

According to the present invention, the validity of the resultant of the amplification reaction of the nucleic acid of the gut microorganism may be determined by the resultant of the amplification reaction of the internal control in the resultant of the amplification reaction.

Typically, the nucleic acid of the bacterium as the internal control selected from the normal gut flora according to the present invention usually needs to be amplified by the amplification reaction and then detected, regardless of the presence or absence of the nucleic acid of the gut microorganism.

According to an embodiment of the present invention, when the nucleic acid of the bacterium as the internal control selected from the normal gut flora is detected, it may be determined that the collection of the sample, the extraction of the nucleic acids, and/or the amplification reaction of the nucleic acids has successfully been executed, and the resultant of the amplification reaction of the nucleic acid of the gut microorganism may be determined to be valid.

According to an embodiment of the present invention, when the nucleic acid of the bacterium as the internal control selected from the normal gut flora is not detected, it may be determined that there was a problem in the collection of the sample, the extraction of the nucleic acids, and/or the amplification of the nucleic acids, and the resultant of the amplification reaction of the nucleic acid of the gut microorganism may be determined not to have a valid result, that is, to be invalid.

As used herein, the term “invalid result” refers to a result that is invalidated due to an uninterpretable detection result. A subject, from which a sample has been determined to have an invalid result, may be again subjected to a test for detection starting from the sample collection step.

According to an embodiment of the present invention, when the internal control nucleic acid is not detected in the resultant of the amplification reaction, the resultant of the amplification reaction of the nucleic acid of the gut microorganism may be determined to be invalid.

According to an embodiment of the present invention, when the internal control nucleic acid is detected in the resultant of the amplification reaction, the detection of the nucleic acid of the gut microorganism indicates the presence (positive) of the nucleic acid of the gut microorganism, and the non-detection of the nucleic acid of the gut microorganism in the resultant of the amplification reaction indicates the absence (negative) of the nucleic acid of the gut microorganism.

According to an embodiment of the present invention, when the internal control nucleic acid is not detected in the resultant of the amplification reaction, the result that the nucleic acid of the gut microorganism is not detected may be determined to be invalid.

According to an embodiment of the present invention, when the internal control nucleic acid is not detected in the resultant of the amplification reaction, the result that the nucleic acid of the gut microorganism is detected may be determined to be invalid.

Generally, the amplification efficiency of the nucleic acid varies according to the target nucleic acid, and the amplification efficiency values may differ even in the same tube according to various factors, such as the initial amount of nucleic acid in the sample and the size of the amplicon.

In the case where the nucleic acid of the gut microorganism is present in a relative excess compared with the internal control nucleic acid, an excess of the nucleic acid of the gut microorganism, during the amplification, is amplified to consume necessary reagents (e.g., polymerase and dNTPs), but the amplification efficiency of the internal control nucleic acid relatively deteriorates, and thus the amplification of the internal control nucleic acid may not be detected.

That is, in such a case, regardless of the presence of the internal control nucleic acid in the sample, the nucleic acid of the gut microorganism is amplified, but the amplification of the internal control nucleic acid may not be detected.

Therefore, according to another embodiment of the present invention, with respect to the result that the internal control nucleic acid is not detected and the nucleic acid of the gut microorganism is detected in the resultant of the amplification reaction, the nucleic acid of the gut microorganism may be determined to be present (positive).

According to an embodiment of the present invention, the method of the present invention may be implemented by, after the collection of the sample, adding an exogenous internal control, besides the above-described internal control, to the collected sample, and may be implemented by including an external positive control and/or an external negative control in the nucleic acid amplification step. The exogenous internal control may be used as a control for the nucleic acid extraction process; the external positive control may be used as a control for the nucleic acid amplification process; and the external negative control may be used as a control for sample contamination and non-specific reactions. For example, an exogenous internal control together with the endogenous internal control according to the present invention may be added to the sample after the collection of the sample, and when the exogenous internal control is detected and the internal control according to the present invention is not detected, the collection of the sample may be determined to have failed.

In accordance with another aspect of the present invention, there is provided a composition for amplifying a nucleic acid of a gut microorganism in a sample using a nucleic acid of a bacterium as an internal control nucleic acid selected from a normal gut flora, the composition including:

(i) a pair of primers for amplifying the nucleic acid of the gut microorganism; and

(ii) a pair of primers for amplifying the nucleic acid of the bacterium selected from the normal gut flora.

The “composition for amplifying a nucleic acid of a gut microorganism in a sample using a nucleic acid of a bacterium as an internal control nucleic acid from a normal gut flora” according to another aspect of the present invention is prepared in order to execute the “method for detecting a nucleic acid of a gut microorganism in a sample using a nucleic acid of a bacterium as an internal control nucleic acid from a normal gut flora” according to an aspect of the present invention, and thus descriptions of overlapping contents therebetween are omitted to avoid excessive complication of the specification due to repetitive descriptions thereof.

In the present invention, the above-described composition of the present invention may selectively include reagents necessary for performing a target amplification reaction (e.g., PCR reaction), such as buffers, DNA polymerase cofactors, and deoxyribonucleotide-5-triphosphate. Optionally, the composition of the present invention may also include various polynucleotide molecules, transcriptase, various buffers and reagents, and antibodies that inhibit DNA polymerase activity. In addition, the composition may include reagents necessary for performing reactions for positive and negative controls. The optimum amounts of reagents used in a particular reaction may be easily determined by a person skilled in the art acquiring the disclosure of the present specification. Typically, the composition of the present invention is prepared in a separate package or compartment comprising the above-mentioned components.

### Advantageous Effects

Features and advantages of the present invention are summarized as follows.

(a) In the method for detecting a nucleic acid of a gut microorganism in a sample according to the present invention, a nucleic acid of a bacterium selected from a normal gut flora is used as an internal control nucleic acid.

(b) The internal control according to the present invention may be used as an internal control for the sample collection process, an internal control for the nucleic acid extraction process, and an internal control for the nucleic acid amplification process.

(c) The internal control according to the present invention is present in the sample from the beginning, and thus there is no inconvenience of separately adding an internal control after the sample collection process.

(d) According to the present invention, the presence or absence of the nucleic acid of the gut microorganism in the sample may be detected with a high accuracy through the minimization of false-negative and false-positive determinations by using the nucleic acid of the bacterium as the internal control selected from the normal gut flora.

### BRIEF DESCRIPTION OF THE DRAWINGS

FIG. 1 shows SEQ ID NO: 1 and SEQ ID NO: 2.

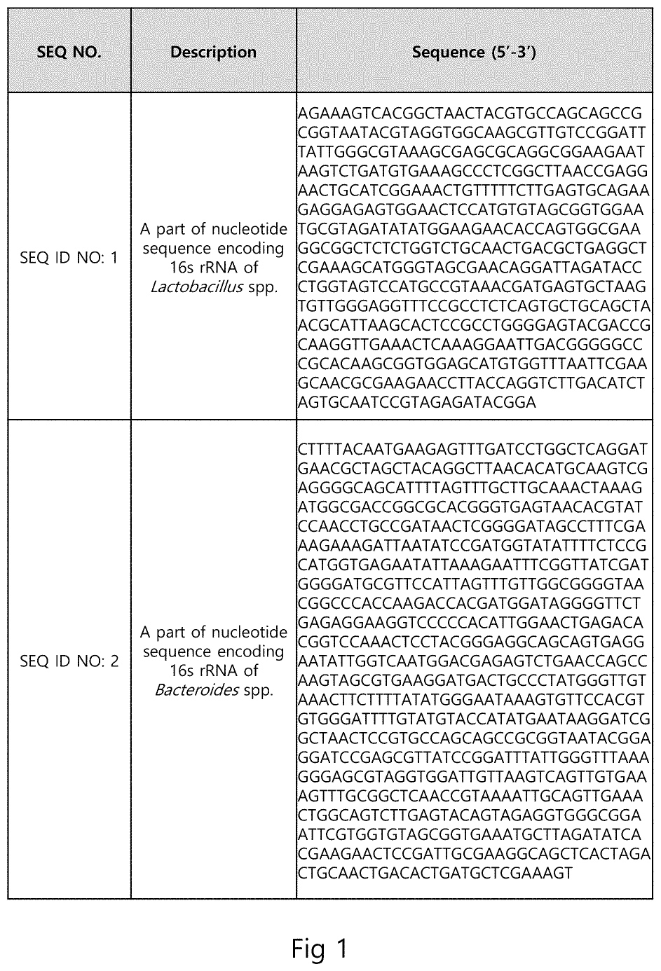

FIG. 2a shows Ct values as detection results of nucleic acids of Lactobacillus spp. and HBB for 24 rectal swab samples collected using eNAT™.

FIG. 2b shows Ct values as detection results of nucleic acids of Lactobacillus spp. and HBB for 48 rectal swab samples collected using Fecal Swab™.

FIG. 2c shows the detection rates (%) and average Ct values of nucleic acids of Lactobacillus spp. and HBB according to the results shown in FIGS. 1a and 1b.

FIG. 3a shows Ct values as detection results of nucleic acids of Bacteroides spp. and HBB for 32 rectal swab samples.

FIG. 3b shows Ct values as detection results of nucleic acids of Bacteroides spp. and HBB for 40 rectal stool samples.

FIG. 3c shows the detection rates (%) and average Ct values of nucleic acids of Bacteroides spp. and HBB according to the results shown in FIGS. 2a and 2b.

FIG. 4 shows the detection results in VanR positive sample and VanR negative samples employing the detection method according to the present invention using a nucleic acid of Lactobacillus spp. as an internal control.

FIG. 5 shows the detection results in VanR positive sample and VanR negative samples employing the detection method according to the present invention using a nucleic acid of Bacteroides spp. as an internal control.

Hereinafter, the present invention will be described in detail with reference to examples. It would be obvious to those skilled in the art that these examples are intended to be more concretely illustrative and the scope of the present invention as set forth in the appended claims is not limited to or by the examples.

### EXAMPLES

### Example 1: Confirmation of Detection Rates of Normal Gut Flora in Various Samples

The present inventors investigated whether nucleic acids of Lactobacillus spp. and Bacteroides spp., which are bacteria selected from a normal gut flora, may be used as internal control nucleic acids in the gut microorganism detection method using nucleic acid amplification.

To this end, the present inventors checked the detection rates of nucleic acids of Lactobacillus spp. and Bacteroides spp. in samples collected from a plurality of persons, and compared the checked detection rates with the detection rate of human beta globin (HBB) gene used as a conventional endogenous internal control.

### Example 1-1: Preparation of Oligonucleotides

As target nucleic acids of Lactobacillus spp. and Bacteroides spp., nucleic acids encoding 16s rRNAs thereof were used. The detection of the nucleic acids employed the TOCE™ technique whereby a plurality of targets may be detected using signals by duplexes formed depending on the presence of target nucleic acid sequences (WO 2012/096523).

Sequences of pairs of primers for detecting target nucleic acids encoding 16s rRNAs of Lactobacillus spp. and Bacteroides spp., probing and tagging oligonucleotide (PTO), and capturing and templating oligonucleotide (CTO) are shown in Table 1. For HBB, a pair of primers for HBB detection (5-globin (human) primer set (Cat. No. 3868; TAKARA)) and PTO and CTO designed based on the pair of primers were used.

TABLE 1
<Example 1-1> Oligonucleotides of composition for
nucleic acid amplification

| | SEQ | Oligo- | |
|---|---|---|---|
| Normal gut | ID | nucleotide | |
| flora | NO | type | Sequence (5′-3′) |
| Lactobacillus | 3 | Forward | CTTGAGTGCAGAAGAGGASAGTG |
| spp. | | primer | |
| Lactobacillus | 4 | Reverse | CRGCACTGAGAGGCGGAAAC |
| spp. | | primer | |
| Lactobacillus | 5 | PTO | GTCGACAGTAGGGGTCTGYAACTGACGCTGAGGCTC |
| spp. | | | [C3 spacer] |
| Lactobacillus | 6 | CTO | [BHQ-2] |
| spp. | | | TTTTATTTATTTTTTTTTTT[T(Quasar670)] |
| | | | TCACCCCTACTGTCGAC[C3 spacer] |
| Bacteroides | 7 | Forward | GGGGATGCGTTCCATTAG |
| Spp. | | primer | |
| Bacteroides | 8 | Reverse | CAATATTCCTCACTGCTGCC |
| Spp | | primer | |
| Bacteroides | 9 | PTO | CATAGGGTTGGCGGGTTCTGAGAGGAAGGTCCCCCAC |
| Spp | | | [C3 spacer] |
| Bacteroides | 10 | CTO | [BHQ-2] |
| Spp | | | TTTTATTTATTTATTTTTTT[T(Quasar670)] |
| | | | TTCCCGCCAACCCTATG[C3 spacer] |

### Example 1-2: Comparison of Detection Rates Between Lactobacillus spp. and HBB

The extraction of nucleic acids was performed using the extraction automation equipment Microlab NIMBUS IVD (Cat. No. 65415-02, Hamilton) and the extraction reagent STARMag 96X4 Universal Cartridge Kit (Cat. No. 744300.4.UC384, Seegene Inc.) for 24 rectal swab samples collected using eNAT™ (eNAT™ PM 2ML REGULAR APPLICATOR; Copan) and 48 rectal swab samples collected using Fecal Swab™ (Cat. No. 480CE; Copan) for sexually transmitted disease testing or gut-related disease testing. Each sample for nucleic acid extraction was used in a volume of 200 μl, and an eluent was used in a volume of 100 μl. The obtained nucleic acid extract was used for real-time polymerase chain reaction.

Taq DNA polymerase having 5′-nuclease activity was used for the extension of forward and reverse primers, the cleavage of PTO, and the extension of CTO.

For the comparison of detection rates between the target nucleic acid of Lactobacillus spp. and the HBB gene in the nucleic acid extract obtained from the same sample, two tubes each containing 5 μl of the nucleic acid extract were prepared. In a first tube (tube 1) of the two tubes were placed 4 pmol of the forward primer (SEQ ID NO: 3) and 4 pmol of the reverse primer (SEQ ID NO: 4) for amplifying a target nucleic acid encoding Lactobacillus spp. 16s rRNA, 4 pmol of PTO (SEQ ID NO: 5), and 1 pmol of CTO (SEQ ID NO: 6), and in a second tube (tube 2) of the two tubes were placed oligonucleotides (forward and reverse primers, PTO, and CTO) for HBB detection in the same amounts as the oligonucleotides for Lactobacillus spp. detection. In each of the two tubes, 5 μl of 4×enzyme mixture [ultimately 3.2 mM dNTPs, 3.2 mM MgCl₂, and 4U Taq DNA polymerase] and 5 μl of 4×enzyme buffer [ultimately 0.04% BSA] were added to prepare a reaction mixture with a final volume of 20 μl. The prepared reaction mixtures were used to perform real-time PCR. The tubes containing the reaction mixtures were placed in a real-time thermocycler (CFX96, Bio-Rad), and then the reaction mixtures were subjected to denaturation at 90° C. for 15 min followed by 45 cycles of 10 sec at 95° C. 15 sec at 60° C. and 10 sec at 72° C. The detection of signals was performed at 60° C. every cycle.

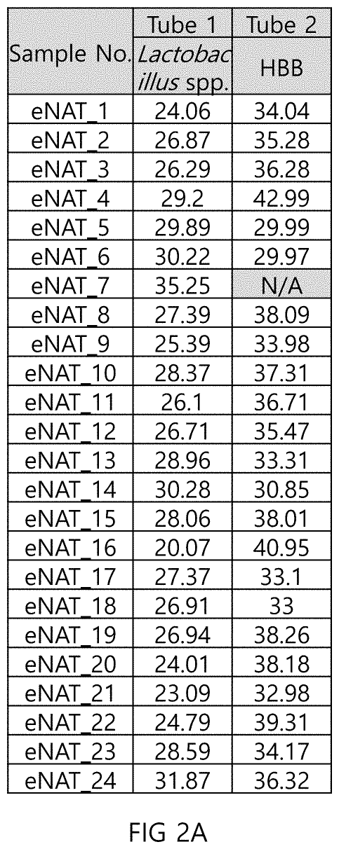

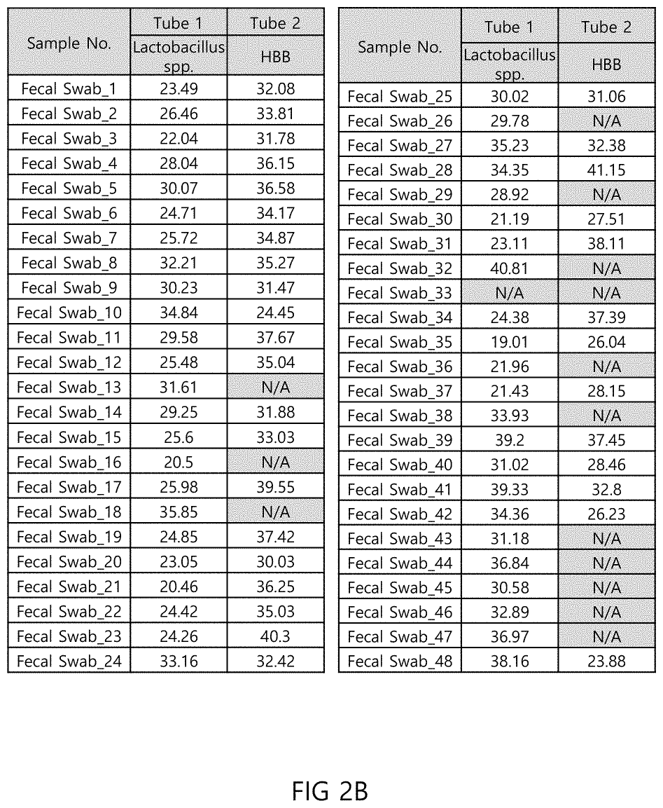

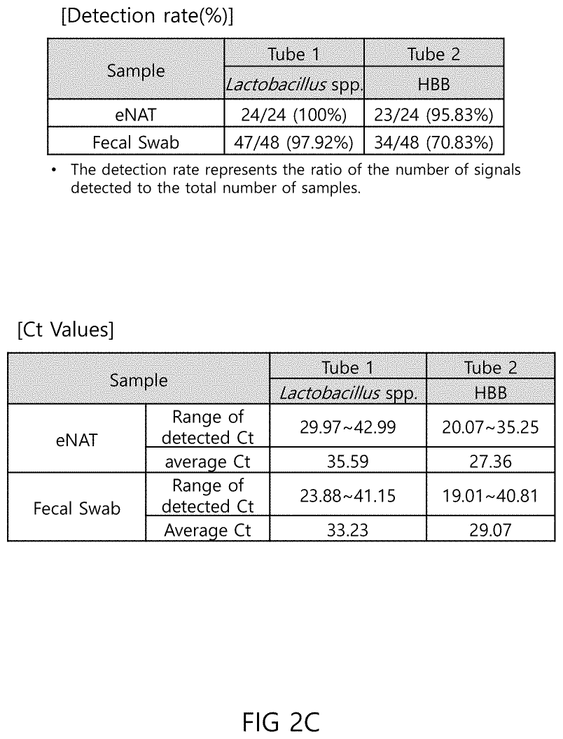

As a result, it was confirmed as shown in FIGS. 2a-2c that the detection rate was 100% for Lactobacillus spp. and 95.83% for HBB in the rectal swab samples collected using eNAT™, and the detection rate was 97.92% for Lactobacillus spp. and 70.83% for HBB in the rectal swab samples collected using Fecal Swab™. It was also confirmed that the average Ct value of Lactobacillus spp. was 27.36 for the rectal swab samples collected using eNAT™ and 29.07 for the rectal swab samples collected using Fecal Swab™, whereas the average Ct value of HBB was 35.59 for the rectal swab samples collected using eNAT™ and 33.23 for the rectal swab samples collected using Fecal Swab™, and thus the average Ct value of Lactobacillus spp. was lower than the average Ct value of HBB.

These results mean that the nucleic acid of Lactobacillus spp., as an internal control, has an excellent detection rate at a more stable level (that is, having a lower Ct value than HBB) compared with the HBB gene, indicating that the nucleic acid of Lactobacillus spp. can be more favorably used as an internal control than the HBB gene, which is generally frequently used as an internal control.

### Example 1-3: Comparison of Detection Rates Between Bacteroides spp. and HBB

The extraction of nucleic acids was performed using the extraction automation equipment Microlab NIMBUS IVD (Cat. No. 65415-02, Hamilton) and the extraction reagent STARMag 96X4 Universal Cartridge Kit (Cat. No. 744300.4.UC384, Seegene Inc.) for 40 stool samples and 42 rectal swab samples (collected using Fecal Swab™) for sexually transmitted disease testing or gut-related disease testing. The stool samples were additionally subjected to a pretreatment step, and for the pretreatment of the stool samples, about 100-200 mg of stool was disintegrated in 1 mL of ASL buffer (Cat. No. 19082, Qiagen), followed by being incubation at room temperature for 10 min, and then a supernatant obtained by centrifugation at 20,000×g (14,000 rpm) for 2 min was used. Each sample for nucleic acid extraction was used in a volume of 200 μl, and an eluent was used in a volume of 100 μl for the rectal swab samples and 50 μl for the stool samples. The obtained nucleic acid extract was used for real-time polymerase chain reaction.

Taq DNA polymerase having 5′-nuclease activity was used for the extension of forward and reverse primers, the cleavage of PTO, and the extension of CTO.

For the comparison of detection rates between the target nucleic acid of Bacteroides spp. and the HBB gene in the nucleic acid extract obtained from the same sample, two tubes each containing 5 μl of the nucleic acid extract were prepared. In a first tube (tube 1) of the two tubes were placed 4 pmol of the forward primer (SEQ ID NO: 7) and 4 pmol of the reverse primer (SEQ ID NO: 8) for amplifying a target nucleic acid encoding Bacteroides spp. 16s rRNA, 4 pmol of PTO (SEQ ID NO: 9), and 1 pmol of CTO (SEQ ID NO: 10), and in a second tube (tube 2) of the two tubes were placed oligonucleotides (forward and reverse primers, PTO, and CTO) for HBB detection in the same amounts as the oligonucleotides for Bacteroides spp. detection. In each of the two tubes, 5 μl of 4×enzyme mixture [ultimately 3.2 mM dNTPs, 3.2 mM MgCl₂, and 4U Taq DNA polymerase] and 5 μl of 4×enzyme buffer [ultimately 0.04% BSA] were added to prepare a reaction mixture with a final volume of 20 μl. The prepared reaction mixtures were used to perform real-time PCR. The tubes containing the reaction mixtures were placed in a real-time thermocycler (CFX96, Bio-Rad), and then the reaction mixtures were subjected to denaturation at 90° C. for 15 min followed by 45 cycles of 10 sec at 95° C. 15 sec at 60° C. and 10 sec at 72° C. The detection of signals was performed at 60° C. every cycle.

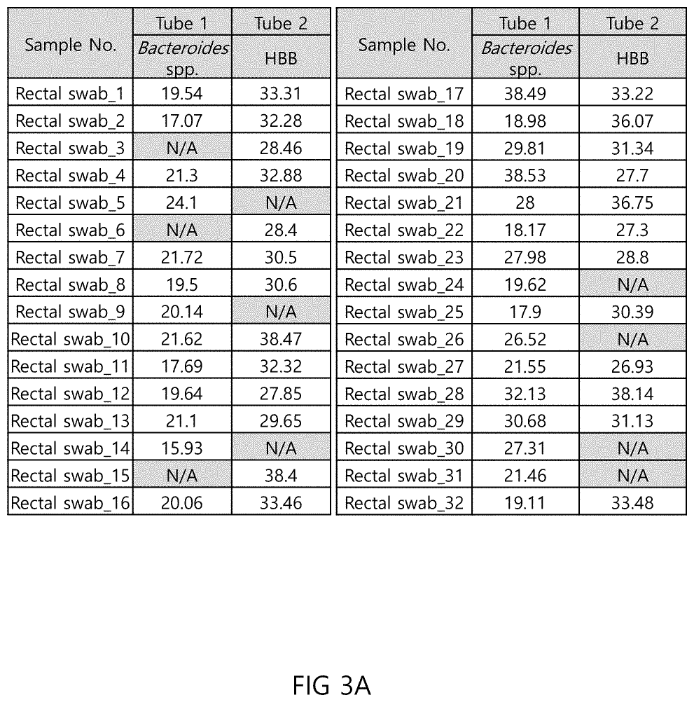

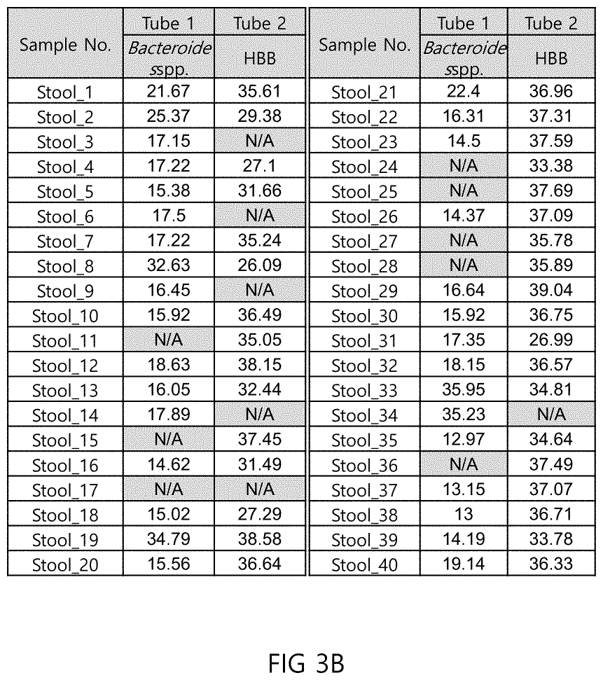

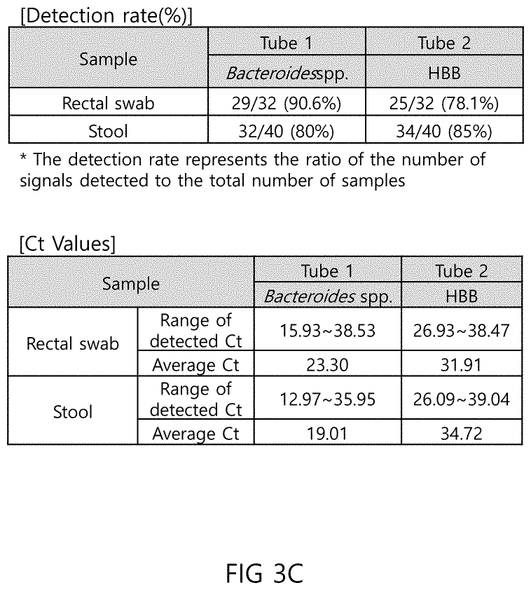

As a result, as shown in FIGS. 3a-3c, for the rectal swab samples, the detection rate of Bacteroides spp. was 90.6% and the average Ct value therefor was 23.30, and the detection of HBB was 78.1% and the average Ct value thereof was 31.91. For the stool samples, the detection rate of Bacteroides spp. was 80% and the average Ct value therefor was 19.01 and the detection of HBB was 85%, which was somewhat higher than that of Bacteroides spp., but the average Ct value therefor was 34.72, which was higher than that of Bacteroides spp.

These results mean that the nucleic acid of Bacteroides spp., as an internal control, also has an excellent detection rate at a more stable level (that is, having a lower Ct value than HBB) compared with the HBB gene, indicating that the nucleic acid of Bacteroides spp. can be more favorably used as an internal control than the HBB gene, which is generally frequently used as an internal control.

### Example 2: Use of Normal Gut Flora as Internal Control

The present inventors detected, as internal controls, nucleic acids of Lactobacillus spp. and Bacteroides spp., which are bacteria selected from a normal gut flora, together with the detection of a nucleic acid of a gut microorganism.

The present inventors detected vancomycin resistant Enterococci (VRE) as gut microorganisms, and used the oligonucleotides contained in Anyplex™ VanR Real-time Detection product (Seegene Inc., Korea) as the oligonucleotides for detecting a nucleic acid of vancomycin resistant Enterococci and the same oligonucleotides as used in example 1 above as the oligonucleotides for detecting an internal control nucleic acid.

### Example 2-1: Use of Lactobacillus Spp. as Internal Control

For objectivity and clarity of the present test, rectal swab samples, diagnosis results of which were confirmed for the presence or absence of vancomycin resistant Enterococci (VRE) by performing a phenotypic antibiotic susceptibility test, which is a gold standard method for drug resistance, and single PCR, were collected and used.

The extraction of nucleic acids from the collected rectal swab samples was performed using the extraction automation equipment Microlab NIMBUS IVD (Cat. No. 65415-02, Hamilton) and the extraction reagent STARMag 96X4 Universal Cartridge Kit (Cat. No. 744300.4.UC384, Seegene Inc.). Each sample for nucleic acid extraction was used in a volume of 200 μl, and an eluent was used in a volume of 100 μl. The obtained nucleic acid extract was used for real-time polymerase chain reaction.

Taq DNA polymerase having 5′-nuclease activity was used for the extension of forward and reverse primers, the cleavage of PTO, and the extension of CTO.

For 5 μl of each of the nucleic acid extracts obtained from samples confirmed for the presence of vancomycin resistant Enterococci and samples confirmed for the absence of vancomycin resistant Enterococci, (i) 4 pmol of the forward primer (SEQ ID NO: 3) and 4 pmol of the reverse primer (SEQ ID NO: 4) for amplifying a target nucleic acid encoding Lactobacillus spp. 16s rRNA, 4 pmol of PTO (SEQ ID NO: 5), and 1 pmol of CTO (SEQ ID NO: 6), and (ii) oligonucleotides (forward and reverse primers, PTO, and CTO) for detecting the nucleic acid of vancomycin resistant Enterococci in the same amounts as the oligonucleotides for Lactobacillus spp. were added, and 5 μl of 4×enzyme mixture [ultimately 3.2 mM dNTPs, 3.2 mM MgCl₂, and 4U Taq DNA polymerase] and 5 μl of 4×enzyme buffer [ultimately 0.04% BSA] were added, to prepare reaction mixtures with a final volume of 20 μl. The prepared reaction mixtures were used to perform real-time PCR. The tubes containing the reaction mixtures were placed in a real-time thermocycler (CFX96, Bio-Rad), and then the reaction mixtures were subjected to denaturation at 95° C. for 15 min followed by 45 cycles of 10 sec at 95° C., 15 sec at 60° C., and 10 sec at 72° C. The detection of signals was performed at 60° C. every cycle.

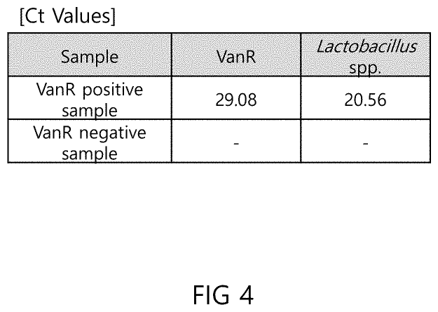

As a result, as shown in FIG. 4, the nucleic acid of vancomycin resistant Enterococci (Ct value: 29.08) and the nucleic acid of Lactobacillus spp. (Ct value: 20.56) were detected for the samples confirmed for the presence of vancomycin resistant Enterococci.

However, the nucleic acid of vancomycin resistant Enterococci and the nucleic acid of Lactobacillus spp. were not detected for the samples confirmed for the absence of vancomycin resistant Enterococci. The non-detection of the internal control indicates that there was a problem in the sample collection process, the nucleic acid extraction process, or the nucleic acid amplification process, and as a result of carrying out an aerobic culture of the corresponding samples, it was confirmed that there was no bacteria growing in media. This indicates that the collection has not been normally executed in the sample collection process, and since the internal control nucleic acid was not detected, the target nucleic acid detection result may be determined to be an invalid result.

These test results verify that the nucleic acid of Lactobacillus spp. can be favorably used as an internal control in the procedure of detecting vancomycin resistant Enterococci.

### Example 2-2: Use of Bacteroides Spp. as Internal Control

For objectivity and clarity of the present test, rectal swab samples, of which diagnosis results were confirmed for the presence or absence of vancomycin resistant Enterococci (VRE) by performing a phenotypic antibiotic susceptibility test, which is a gold standard method for drug resistance, and single PCR, were collected and used.

The extraction of nucleic acids from the collected rectal swab samples was performed using the extraction automation equipment Microlab NIMBUS IVD (Cat. No. 65415-02, Hamilton) and the extraction reagent STARMag 96X4 Universal Cartridge Kit (Cat. No. 744300.4.UC384, Seegene). Each sample for nucleic acid extraction was used in a volume of 200 μl, and an eluent was used in a volume of 100 μl. The obtained nucleic acid extract was used for real-time polymerase chain reaction.

Taq DNA polymerase having 5′-nuclease activity was used for the extension of forward and reverse primers, the cleavage of PTO, and the extension of CTO.

For 5 μl of each of the nucleic acid extracts obtained from samples confirmed for the presence of vancomycin resistant Enterococci and samples confirmed for the absence of vancomycin resistant Enterococci, (i) 4 pmol of the forward primer (SEQ ID NO: 7) and 4 pmol of the reverse primer (SEQ ID NO: 8) for amplifying a target nucleic acid encoding Bacteroides spp. 16s rRNA, 4 pmol of PTO (SEQ ID NO: 9), and 1 pmol of CTO (SEQ ID NO: 10), and (ii) oligonucleotides (forward and reverse primers, PTO, and CTO) for detecting the nucleic acid of vancomycin resistant Enterococci in the same amounts as the oligonucleotides for Bacteroides spp. were added, and 5 μl of 4×enzyme mixture [ultimately 3.2 mM dNTPs, 3.2 mM MgCl₂, and 4U Taq DNA polymerase] and 5 μl of 4×enzyme buffer [ultimately 0.04% BSA] were added, to prepare reaction mixtures with a final volume of 20 μl. The prepared reaction mixtures were used to perform real-time PCR. The tubes containing the reaction mixtures were placed in a real-time thermocycler (CFX96, Bio-Rad), and then the reaction mixtures were subjected to denaturation at 95° C. for 15 min followed by 45 cycles of 10 sec at 95° C., 15 sec at 60° C., and 10 sec at 72° C. The detection of signals was performed at 60° C. every cycle.

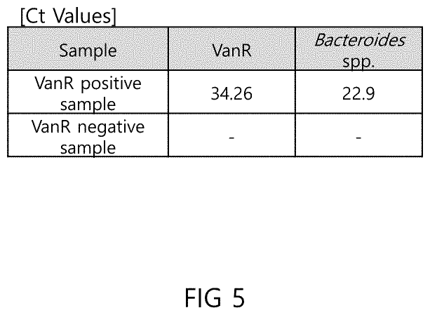

As a result, as shown in FIG. 5, the nucleic acid of vancomycin resistant Enterococci (Ct value: 34.26) and the nucleic acid of Bacteroides spp. (Ct value: 22.9) were detected for the samples confirmed for the presence of vancomycin resistant Enterococci.

However, the nucleic acid of vancomycin resistant Enterococci and the nucleic acid of Bacteroides spp. were not detected for the samples confirmed for the absence of vancomycin resistant Enterococci. The non-detection of the internal control indicates that there was a problem in the sample collection process, the nucleic acid extraction process, or the nucleic acid amplification process, and as a result of carrying out an aerobic culture of the corresponding samples, it was confirmed that there was no bacteria growing in media. This indicates that the collection has not been normally executed in the sample collection process, and since the internal control nucleic acid was not detected, the target nucleic acid detection result may be determined to be an invalid result.

These results verify that the nucleic acid of Bacteroides spp. can be favorably used as an internal control in the procedure of detecting vancomycin resistant Enterococci.

Therefore, it can be seen from the above test results that nucleic acids from a normal gut flora, such as Lactobacillus spp. and Bacteroides spp., can be favorably used as internal controls in the gut microorganism detection method using nucleic acid amplification.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for detecting a nucleic acid of a gut microorganism in a sample using a nucleic acid of a bacterium as an internal control nucleic acid selected from a normal gut flora, the method comprising:
collecting and preparing the sample;
performing an amplification reaction of a nucleic acid in the sample using (i) a pair of primers for amplifying the nucleic acid of the gut microorganism; and (ii) a pair of primers for amplifying the nucleic acid of the bacterium as the internal control nucleic acid selected from the normal gut flora;
detecting a resultant of the amplification reaction;
determining a validity of the amplification reaction of the nucleic acid of the gut microorganism by using a resultant of the amplification reaction of the internal control nucleic acid; and
determining whether the nucleic acid of the gut microorganism is present or not in the sample by (i) the determined validity and (ii) the resultant of the amplification reaction of the nucleic acid of the gut microorganism.

2. The method according to claim 1, wherein the preparation of the sample comprises extracting nucleic acids from the sample.

3. The method according to claim 2, wherein the nucleic acid of the bacterium selected from the normal gut flora is used as an internal control nucleic acid for the steps of collecting the sample, extracting the nucleic acids from the sample and/or amplifying the extracted nucleic acids.

4. (canceled)

5. (canceled)

6. The method according to claim 1, wherein the resultant of the amplification reaction of the nucleic acid of the gut microorganism is determined to be invalid when the internal control nucleic acid is not detected.

7. The method according to claim 1, wherein the normal gut flora is a human normal gut flora.

8. The method according to claim 1, wherein the bacterium selected from the normal gut flora is Bacteroides spp. or Lactobacillus spp.

9. The method according to claim 1, wherein the nucleic acid of the bacterium selected from the normal gut flora comprises a nucleotide sequence encoding 16s rRNA.

10. The method according to claim 1, wherein the sample is a rectal swab sample, a stool sample, or a urine sample.

11. The method according to claim 1, wherein the detecting of the resultant of the amplification reaction is performed in a post-amplification detection manner or in a real-time detection manner.

12. The method according to claim 1, wherein the amplification reaction further comprises (i) a probe for detecting the nucleic acid of the gut microorganism; and (ii) a probe for detecting the nucleic acid of the bacterium selected from the normal gut flora.

13. The method according to claim 12, wherein the pair of primers or the probe for the nucleic acid of the bacterium selected from the normal gut flora comprises a nucleotide sequence that specifically hybridizes with a nucleotide sequence selected from the group consisting of the sequences of SEQ ID NO:1 and 2 and complementary sequences thereof.

14. The method according to claim 1, wherein the amplification reaction is performed by a fast PCR method.

15. The method according to claim 1, wherein the gut microorganism is a drug-resistant gut microorganism.

16. A composition for amplifying a nucleic acid of a gut microorganism in a sample using a nucleic acid of a bacterium as an internal control nucleic acid selected from a normal gut flora, the composition comprising:
(i) a pair of primers for amplifying the nucleic acid of the gut microorganism; and
(ii) a pair of primers for amplifying the nucleic acid of the bacterium selected from the normal gut flora.

17. The composition according to claim 16, wherein the nucleic acid of the bacterium selected from normal gut flora is used as an internal control nucleic acid for the steps of collecting the sample, extracting nucleic acids from the sample and/or amplifying the extracted nucleic acids.

18. The composition according to claim 16, wherein the normal gut flora is a human normal gut flora.

19. The composition according to claim 16, wherein the bacterium selected from the normal gut flora is Bacteroides spp. or Lactobacillus spp.

20. The composition according to claim 16, wherein the nucleic acid of the bacterium selected from the normal gut flora comprises a nucleotide sequence encoding 16s rRNA.

21. The composition according to claim 16, wherein the composition further comprises (i) a probe for detecting the nucleic acid of the gut microorganism; and (ii) a probe for detecting the nucleic acid of the bacterium selected from the normal gut flora.

22. The composition according to claim 21, wherein the pair of primers or the probe for the nucleic acid of the bacterium selected from the normal gut flora comprises a nucleotide sequence that specifically hybridizes with a nucleotide sequence selected from the group consisting of the sequences of SEQ ID No: 1, SEQ ID No: 2 and complementary sequences thereof.

23. The composition according to claim 16, wherein the gut microorganism is a drug-resistant gut microorganism.


SEQUENCE LISTING (10 sequences)

SEQ ID NO: 1 - DNA, 529 bp, Lactobacillus sp.
agaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacgga

SEQ ID NO: 2 - DNA, 776 bp, Bacteroides sp.
cttttacaatgaagagtttgatcctggctcaggatgaacgctagctacaggcttaacacatgcaagtcgaggggcagcattttagtttgcttgcaaactaaagatggcgaccggcgcacgggtgagtaacacgtatccaacctgccgataactcggggatagcctttcgaaagaaagattaatatccgatggtatattttctccgcatggtgagaatattaaagaatttcggttatcgatggggatgcgttccattagtttgttggcggggtaacggcccaccaagaccacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgttccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagt

SEQ ID NO: 3 - DNA, 23 bp, Artificial Sequence
  Forward Primer for detecting Lactobacillus sp.
cttgagtgcagaagaggasagtg

SEQ ID NO: 4 - DNA, 20 bp, Artificial Sequence
  Reverse Primer for detecting Lactobacillus sp.
crgcactgagaggcggaaac

SEQ ID NO: 5 - DNA, 36 bp, Artificial Sequence
  PTO for detecting Lactobacillus sp.
gtcgacagtaggggtctgyaactgacgctgaggctc

SEQ ID NO: 6 - DNA, 38 bp, Artificial Sequence
  CTO for detecting Lactobacillus sp.
ttttatttatttttttttttttcacccctactgtcgac

SEQ ID NO: 7 - DNA, 18 bp, Artificial Sequence
  Forward Primer for detecting Bacteroides sp.
ggggatgcgttccattag

SEQ ID NO: 8 - DNA, 20 bp, Artificial Sequence
  Reverse Primer for detecting Bacteroides sp.
caatattcctcactgctgcc

SEQ ID NO: 9 - DNA, 37 bp, Artificial Sequence
  PTO for detecting Bacteroides sp.
catagggttggcgggttctgagaggaaggtcccccac

SEQ ID NO: 10 - DNA, 38 bp, Artificial Sequence
  CTO for detecting Bacteroides sp.
ttttatttatttattttttttttcccgccaaccctatg